(19) United States
(12) Patent Application Publication
(10) Pub. No.: US 2022/0193043 A1
(43) Pub. Date: Jun. 23, 2022

(54) Pharmaceutical Combination Formulations Comprising Tizanidine, Resveratrol and Piperine

(71) Applicant: CIPLA LIMITED, Mumbai (IN)

(72) Inventors: Kalpana JOSHI, Thane (IN); Jeevan GHOSALKAR, Thane (IN); Deepak Dnyaneshwar GAIKWAD, Mumbai (IN)

(21) Appl. No.: 17/599,391

(22) PCT Filed: Mar. 27, 2020
(86) PCT No.: PCT/IN2020/050294

§ 371 Date: Sep. 28, 2021

(30) Foreign Application Priority Data
Mar. 29, 2019 (IN) 201921012764

(51) Int. Cl. A61K 31/428; A61K 47/10; A61K 47/46; A61K 47/22

(52) U.S. Cl. CPC A61K 31/428; A61K 47/22; A61K 47/46; A61K 47/10

(57) ABSTRACT

The present invention relates to method of increasing the bioavailability/bio-efficacy of tizanidine by co-administering with resveratrol and bioenhancer. The formulation comprising tizanidine, resvetarol and bioenhancer are also provided which can be used for treatment of muscle spasticity.

### CROSS REFERENCE TO RELATED APPLICATIONS

This application is a filing under 35 U.S.C. 371 of International Application No. PCT/IN2020/050294, filed Mar. 27, 2020, entitled “Pharmaceutical Combination Formulations Comprising Tizanidine, Resveratrol and Piperine,” which claims priority to Indian Patent Application No. 201921012764, filed Mar. 29, 2019, which applications are incorporated by reference herein in their entirety.

### FIELD OF INVENTION

The present invention relates to method of enhancing bioavailability and bio-efficacy of tizanidine. It relates to enhancement of bioavailability of tizanidine by co-administration of the drug with resveratrol and at least one bioenhancer. The present invention has also provided pharmaceutical formulations of tizanidine, resveratrol and bioenhancer and optionally pharmaceutically acceptable excipients. The present invention further relates to process for preparing pharmaceutical formulations comprising tizanidine, resveratrol and/or at least one bioenhancer.

### BACKGROUND OF INVENTION

Cerebral palsy results from a non-progressive injury to the developing central nervous system and produces motor dysfunction, movement disorders, mental deficits and impaired function. Although the CNS lesion occurs once and remains constant, expression of this lesion is affected by the interactions of growth, development, maturation and disease processes that may confound the clinical picture.

Motor dysfunction associated with cerebral palsy may include spasticity, rigidity and weakness. Spasticity is a common syndrome occurring in over 80% of cerebral palsy patients. It is characterized by increased muscle tone, resistance/difficulty in extending muscles, and excessive activation of skeletal muscles (such as spasms and exaggerated tendon jerks) due to hyperexcitability of the stretch reflex. Additionally, spasticity may be accompanied by pain, weakness, fatigue and lack of dexterity. The mechanism of spasticity-related pain is not well understood, but the pain may be associated with spasticity, as well as the resulting impairment and deformity. The increase in muscle tone affects the patients' gait, posture, sleep, and ability to perform everyday activities and makes physiotherapy and nursing care of bedridden patients difficult. If excessive spasticity is untreated, it can lead to tendon contractures, deformities, pain, and significant physical impairment, which have a negative impact on health-related quality of life.

Spasticity is associated with sleep disturbance. It negatively impacts on sleep and causes arousal through the mechanisms of muscle spasm and pain. Muscle spasms cause uncontrolled limb movements of various intensities and pain, either acute pain directly due to the muscle spasm or sub-acute pain due to unrelieved uncomfortable posturing.

Treatment of spasticity maybe divided into two categories: (a) rehabilitative techniques (physiotherapy), and (b) interventional therapy (operative and pharmacologic). Most adult patients are treated with physical therapy alone with little regard to medical treatments available.

Tizanidine is a central alpha-2-adrenergic agonist indicated for the management of spasticity. The structure of tizanidine is:

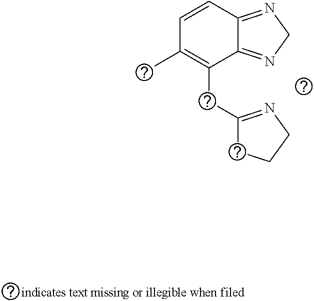 [SMILES: Cc1ccc2c(c1CC1=NCCC1)=NCN=2]

Tizanidine hydrochloride is commercially available in an immediate release oral tablet formulation under the brand name Zanaflex™. It is a conventional oral dosage form whose active ingredient is absorbed into the bloodstream through the mucosa lining the Stomach and Small intestine. Tizanidine hydrochloride may be used to treat particular types of spasticity, such as spasticity in multiple sclerosis, spasticity caused by spinal chord injury, and spasticity caused by stroke or brain injury. The bioavailability of tizanidine is highly variable from patient to patient, necessitating titration of the dose

level on an individual basis. Tizanidine can cause hepatic toxicity, which is another reason that the dosage and plasma level of tizanidine should be carefully controlled. Tizanidine that is administered in Zanaflex™ is essentially completely absorbed by the intestinal mucosa but the bioavailability of tizanidine is only about 40% due to first-pass hepatic metabolism to metabolites, all of which appear to be pharmacologically inactive. Tizanidine is a short-acting drug requiring frequent, multiple daily dosing. The recommended starting dose of Zanaflex is 2 mg; dose can be repeated at 6 to 8 hour intervals, up to a maximum of 3 doses in 24 hours. The dosage can be increased by 2 mg to 4 mg per dose, with 1 to 4 days between increases and total daily dose should not exceed 36 mg. Tizanidine's extensive first-pass hepatic metabolism resulting both in a lowered bioavailability (22-40%), as well as an increased potential for liver toxicity. Further, in vitro results indicated that CYP1A2 is primarily responsible for the metabolism of tizanidine. Increasing the bioavailability of tizanidine by either adding bioenhancers or by stopping the hepatic metabolism or both can help in better management of spasticity, reduction in dose and dosage intervals.

WO2016193880 discloses immediate release pharmaceutical compositions of reTizanidine.

WO2007016676 discloses long acting tizanidine formulations providing a tizanidine blood concentration of at least about 900 pg/ml for about five hours, wherein the formulation is administered prior to bedtime.

US20040122065 proposed pharmaceutical compositions of tizanidine administered by buccal and sublingual delivery to improve the bioavailability of tizanidine

Thus, some strategies and formulations have been employed previously to increase the bioavailability of tizanidine, there still remains a need to improve the systemic absorption of tizanidine and enable it for the management of spasticity.

Bioenhancers are molecules, which do not possess drug activity of their own but promote and augment the biological activity or bioavailability or the uptake of drugs in combination therapy. Natural products especially from plant sources have played an important role in drug development. Either the isolated plant biomolecules or its synthetic, semi synthetic derivatives have provided useful clues in the production of medicines. According to WHO nearly 80% of the world's population relies on herbal medicines as primary health care. Synergism in which the action of one biomolecule is enhanced by another unrelated chemical has been the hallmark of herbal drugs. For instance, as disclosed in Stermitz et al 2000, Proc. Natl. Acad Sci. (USA) 97: 1433-1437, berberine and antimicrobial alkaloid isolated from Berberis fremontii has very weak antimicrobial activity in solution. However, in combination with other compound 5-Methoxy hydnocarpin (5-MIC) the antimicrobial activity of berberine is increased by 200 folds against Staphylococcus aurues. Bioenhancers act by various mechanisms such as P-gp inhibition activity, reduction in acid secretion, increase in gastrointestinal blood supply, inhibition of gastrointestinal transit, gastric emptying, increase in intestinal mortality, modifications in GIT epithelial cell membrane permeability and inhibition of first pass metabolism and metabolizing enzymes. Bioenhancers constitute an innovative concept and judicious use of them can lead to reductions in drug cost, toxicity, and other adverse effects.

Present day research on expensive, toxic and scarce drugs or drugs that exhibit poor bioavailability demands the use of an ideal bioenhancer which should be safe, effective, economical, easily procured, non-addictive etc. Further, bioavailability enhancement helps to lower dosage levels and shorten the treatment course.

Incase of Tizandine, no earlier attempt for improvement of bioavailability with the use of bioenhancers is made. Because of the short duration of therapeutic effect, treatment with tizanidine is reserved for those daily activities and times when relief of spasticity is most important. However, if bioavailability of tizanidine is increased with the help of bioenhancers, it will prove as a huge therapeutic benefit. Looking at this, there is an unmet need for tizanidine formulations with improved bioavailability.

The inventors of present invention studied the effect of bioenhancers on the bioavailability of tizanidine and after rigorous experimentation provided combination formulations of tizanidine with some bioenhancers so that the bioavailability of tizanidine is increased at a desired level, enhancing the therapeutic effect, reducing the dose and shorten the treatment course.

### OBJECT OF INVENTION

The object of present invention is to provide a method for enhancing bioavailability/bio-efficacy of tizanidine by co-administration with resveratrol and bioenhancer. Also the object of present invention is to provide a pharmaceutical composition comprising tizanidine, resveratrol and at least one bioenhancer and optionally pharmaceutically acceptable excipients having improved bioavailability or bioefficacy, solubility and systemic absorption.

Another object of the present invention is to provide a composition comprising tizanidine, resveratrol and at least one bioenhancer and optionally pharmaceutically acceptable excipients for once or twice a day administration.

Yet another object of the present invention is to provide a composition comprising tizanidine, resveratrol and at least one bioenhancer and optionally pharmaceutically acceptable excipients for once or twice a day administration with reduced dose.

Another object of the present invention is to provide a composition comprising tizanidine, resveratrol and at least one bioenhancer and optionally pharmaceutically acceptable excipients in the form of a kit.

Still another object of the present invention is to provide a method for management of spasticity which method comprises administering a pharmaceutical composition comprising tizanidine, resveratrol and at least one bioenhancer.

Yet another object of the present invention is to provide the use of a pharmaceutical composition comprising tizanidine, resveratrol and at least one bioenhancer for the management of spasticity.

### SUMMARY OF INVENTION

According to an aspect of present invention, there is provided a method of enhancing bioavailability/bio-efficacy of tizanidine by co-administration with resveratrol and bioenhancer.

Also another aspect of present invention is to provide a pharmaceutical composition comprising tizanidine, resveratrol and at least one bioenhancer and optionally pharmaceutically acceptable excipients having improved bioavailability or bioefficay, solubility and systemic absorption.

According to another aspect of present invention, there is provided a composition comprising tizanidine, resveratrol and at least one bioenhancer and optionally pharmaceutically acceptable excipients for once or twice a day administration.

According to yet an aspect of present invention, there is provided a composition comprising tizanidine, resveratrol and at least one bioenhancer and optionally pharmaceutically acceptable excipients for once or twice a day administration with reduced dose.

According to another aspect of present invention, there is provided a composition comprising tizanidine, resveratrol and at least one bioenhancer and optionally pharmaceutically acceptable excipients in the form of a kit.

Still another aspect of the present invention is to provide a method for management of spasticity which method comprises administering a pharmaceutical composition comprising tizanidine, resveratrol and at least one bioenhancer and optionally pharmaceutically acceptable excipients.

Yet another aspect of the present invention is to provide the use of a pharmaceutical composition comprising tizanidine, resveratrol and at least one bioenhancer for the management of spasticity.

### BRIEF DESCRIPTION OF THE FIGURES

FIG. 1: Cmax, AUC of group 1, 2, 3

FIG. 2: Plasma concentration of group 1, 2, 3

FIG. 3: Cmax, AUC of group 4, 5, 6

FIG. 4: Plasma concentration of group 4, 5, 6

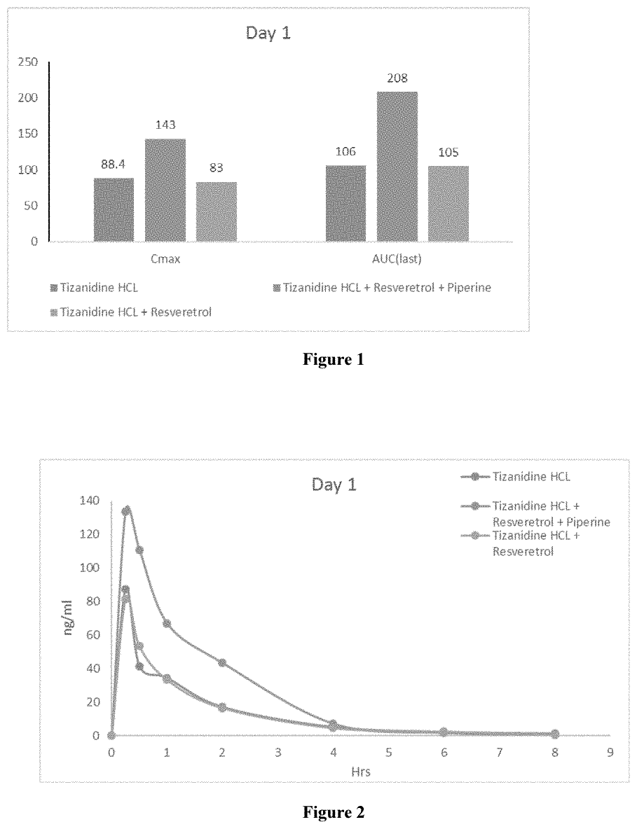

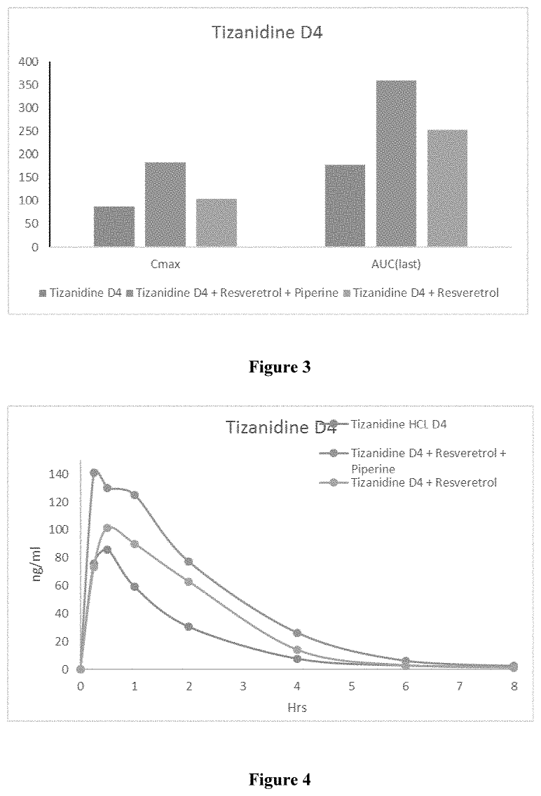

### DETAILED DESCRIPTION OF INVENTION

Due to the oral bioavailability and extensive first pass metabolism, inventors of present invention have made attempts to increase the bioavailability of tizanidine with the use of bioenhancers. Because of increased bioavailability, the dose and cost of active drug can be reduced, making the formulation cheaper and safer, better tolerated, having better efficacy, better compliance.

As used herein, the term “composition”, as in pharmaceutical composition, is intended to encompass a drug product comprising Tizanidine or its pharmaceutically acceptable salts, esters, solvates, polymorphs, stereoisomers or mixtures thereof, and the other inert ingredient(s) (pharmaceutically acceptable excipients). Such pharmaceutical compositions are synonymous with “formulation” and “dosage form”. Pharmaceutical composition of the invention include, but is not limited to, granules, tablets (single layered tablets, multilayered tablets, mini tablets, bioadhesive tablets, caplets, matrix tablets, tablet within a tablet, mucoadhesive tablets, modified release tablets, orally disintegrating tablets, pulsatile release tablets, timed release tablets, delayed release, controlled release, extended release and sustained release tablets), capsules (hard and soft or liquid filled soft gelatin capsules), pills, troches, sachets, powders, microcapsules, minitablets, tablets in capsules and microspheres, matrix composition and the like. Preferably, the pharmaceutical composition refers to tablets and capsules. More preferably, the pharmaceutical composition refers to hard gelatin capsules or HPMC based capsules. Most preferably, the pharmaceutical composition refers to hard gelatin capsules.

The term “excipient” means a pharmacologically inactive component such as a diluent, lubricant, surfactant, carrier, or the like. The excipients that are useful in preparing a pharmaceutical composition are generally safe, non-toxic and are acceptable for veterinary as well as human pharmaceutical use. Reference to an excipient includes both one and more than one such excipient. Co-processed excipients are also covered under the scope of present invention.

The term “bioequivalent” means the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. In practice, two products are considered bioequivalent if the 90% confidence interval of the Cmax, AUC, or, optionally, Tmax is within the range of 80.00% to 125.00%.

The term “an effective amount of an bioenhancer” relates to amount sufficient to enhance the bioavailability or bioefficacy of tizanidine by about 50% to about 150%.

Unless otherwise stated the weight percentages expressed herein are based on the final weight of the composition or formulation.

The inventors of present invention have done experiments of tizanidine with a variety of known bioenhancers such as, lecithin, piperine, garlic, lysergol, and the like and surprisingly, it was observed that use of resveratrol alone and in combination with other bioenhancer can alter the bioavailability of Tizanidine and its salts. Further, in addition it was also found that when tizanidine was co-administered with resveratrol and piperine, the bioavailability of tizanidine increased many folds. The present invention provides pharmaceutical compositions comprising tizanidine, resveratrol and piperine wherein the enhanced bioavailability of tizanidine by piperine is maintained in presence of resveratrol.

The present invention relates to stable pharmaceutical composition of Tizanidine or its pharmaceutically acceptable esters, salts, esters, solvates, polymorphs, stereoisomers or mixtures thereof. Preferably, salt of Tizanidine is Tizanidine Hydrochloride. Tizanidine hydrochloride in the present invention is used in an amount of about 0.1-50% by weight with respect to total weight of the pharmaceutical composition. Preferably, tizanidine hydrochloride in the present invention is used in an amount of about 2-40% by weight with respect to total weight of the pharmaceutical composition.

In some embodiments, tizanidine has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). Thus, in an embodiment of present invention, tizanidine can be present as deuterated tizanidine.

According to the present invention, the dose of tizanidine or its pharmaceutically acceptable salts, esters, solvates, polymorphs, stereoisomers or mixtures thereof, or its deuterated form is reduced by 5-95% as compared to the standard dose of tizanidine.

In an embodiment, the formulation of present invention comprises tizanidine or its pharmaceutically acceptable salts, resveratrol and piperine wherein the bioavailability of tizanidine is increased.

Resveratrol is a polyphenol found in grape skins and peanuts. The chemical structure of resveratrol leads to low water solubility (<0.05 mg/mL), which affects its absorption. At the intestinal level, resveratrol is absorbed by passive diffusion or forming complexes with membrane transporters, such as integrins. Once in the bloodstream, resveratrol can be found essentially in three different forms: glucuronide, sulfate, or free. Resveratrol has a high metabolism, leading to the production of conjugated sulfates. However, the nature and quantity of these metabolites can differ between subjects due to interindividual variability. Suitable sources of resveratrol include, but are not limited to resveratrol derived from natural sources such as grape skins, wine, or other botanical sources such as P. cuspidatum or C. quinquangulata, or produced synthetically such as 98% trans-resveratrol, available commercially from Sigma Chemical Co., St. Louis. Mo. Reseveratrol for use in the compositions can be trans-resveratrol, cis-resveratrol, or a combination thereof. Botanical extracts with higher concentrations of resveratrol may be produced by fractionation and further column chromatography until an extract containing up to a 99% concentration of resveratrol (e.g. as a mixture of both cis- and trans-resveratrol) is produced. Resveratrol has the property to inhibit efflux transport and decrease renal clearance of actives with which it is administered.

The compositions of the invention may also be made using modified resveratrol or derivatives of resveratrol. Some non-limiting examples of resveratrol derivatives for making the compositions of the invention include, but are not limited to those disclosed in EP2774915, WO2010062824, and WO201006282, the disclosures of which are incorporated by reference in their entirety for all purposes.

The amount of resveratrol incorporated in present invention is from about 10% to about 80% of total weight of formulation.

According to the present invention, resveratrol is contacted with (e.g. combined, blended or mixed) with one or more bioenhancers. Such bioenhancers may increase the bioavailability, bioactivity and/or efficacy of resveratrol. Some non-limiting examples of bioenhancers for combining with resveratrol include, but are not limited to, lecithin, piperine, garlic, lysergol, naringin, quercetin, niaziridin, glycyrrhizin, capsaicin, curcumin, ferulic acid and oils such as grape seed oil, peppermint oil, carrot oil, caraway oil, capmul, MCM C10, and combinations thereof. The bioenhancers disclosed herein may be used individually, or in combination with one another.

In specific embodiment of present invention, tizanidine co-adminstered with resveratrol and piperine both to increase the bioavailability of tizanidine.

The compound piperine may be obtained as an extract from the fruit of Piper nigrum. The fruit of black pepper (Piper nigrum L.) and long pepper (Piper longum L.) are both important medicinal herbs Black pepper contains approximately 5-9% piperine and is listed by the FDA as an herb which is generally recognized as safe (GRAS) for its intended use as spice, seasoning, or flavoring. The extract from black pepper has higher concentration of piperine than natural black pepper and extract from Piper longum having a higher concentration of piperine than natural Piper longum.

Piperine is chemically known as (1-2E, 4E-piperinoyl-piperidine) and is structurally represented as below

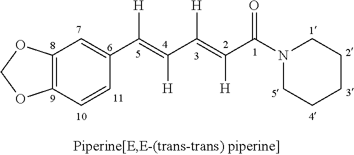 [SMILES: O=C(/C=C/C=C/c1ccc2c(c1)OCO2)N1CCCCC1]

Without being bound to any theory whatsoever, Piperine may increase the drug bioavailability by inhibiting enzymes which participate in the biotransformation of drugs and thus preventing their inactivation and elimination. It also inhibits p-glycoprotein and major drug metabolizing enzyme CYP3A4. the ‘pump’ protein that removes substances from cells and can decrease the intestinal production of glucuronic acid, thereby permitting more substances to enter the body in active form.

Piperine may enhance the drug bioavailability by promoting rapid absorption of drugs and nutrients by increasing blood supply to the gastrointestinal tract, decreasing hydrochloric acid secretion to prevent the breakdown of some drugs, increasing the emulsifying content of the gut, increasing enzymes like γ-glutamyl transpeptidase which participate in active and passive transport of nutrients to the intestinal cells.

Piperine has also been reported to occur in other Piper species i.e. P. acutisleginum, album, argyrophylum, attenuatum, aurantiacum, betle, callosum, chaba, cubeba, guineense, hancei, khasiana, longum, macropodum, nepalense, novae hollandiae, peepuloides, retrokacturn, sylvaticum.

Tetrahydro piperine is a structural analog of Piperine. The two double bonds at position 2 and 4 are saturated to give a tetrahydro analog. Tetrahydropiperine is chemically known as 5-(1,3-benzodioxol-5-yl)-1-piperidin-1-ylpentan-1-one and is structurally represented as below.

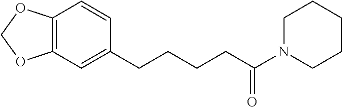 [SMILES: O=C(CCCCc1ccc2c(c1)OCO2)N1CCCCC1]

Tetrahydropiperine occurs like piperine naturally in black pepper (about 0.7% in black pepper oleoresin). Tetrahydropiperine can be synthesized from piperine which is previously extracted from black pepper oleoresin.

The term “analogs or derivatives” of tetrahydropiperine is used in broad sense to include alkyltetrahydropiperines, such as methyltetrahydropiperine or ethyltetrahydropiperine, dialkyltetrahydropiperines, such as dimethyltetrahydropiperine or diethyltetrahydropiperine, alkoxylated tetrahydropiperine, such as methoxy tetrahydropiperine, hydroxylated tetrahydropiperine, e.g. 1-[(5,3-benzodioxyl-5-yl)-1-hydroxy-2,4-pentadienyl]-piperine, 1-[(5,3-benzodioxyl-5-yl)-1-methoxy-2,4-pentadienyl]-piperine, halogenated tetrahydropiperine, such as 1-[(5,3-benzodioxyl-5-yl)-1-oxo-4-halo-2-pentenyl]-piperine and 1-[(5,3-benzodioxyl-5-yl)-1-oxo-2-halo-4-pentenyl]-piperine, dihydropiperine, alkyldihydropiperines, such as methyldihydropiperine or ethyldihydropiperine, dialkyldihydropiperines, such as, dimethyldihydropiperine or diethyldihydropiperine, alkoxylated dihydropiperine such as methoxy dihydropiperine, and halogenated dihydropiperine and their pharmaceutically acceptable salts, pharmaceutically acceptable solvates, pharmaceutically acceptable hydrates, pharmaceutically acceptable anhydrates, pharmaceutically acceptable enantiomers, pharmaceutically acceptable esters, pharmaceutically acceptable isomers, pharmaceutically acceptable polymorphs, pharmaceutically acceptable prodrugs, pharmaceutically acceptable tautomers, pharmaceutically acceptable complexes etc.

In an embodiment of present invention, the piperine used in the present invention may be occurring naturally in the fruits or may be prepared synthetically by the process well known in the art. The piperine or its derivatives prepared synthetically or extracted from the naturally occurring fruits are substantially pure. The term “substantially pure piperine” herein refers to piperine having purity (measured by HPLC) above 99.5%, preferably above about 99.7%, and more preferably above about 99.9%. The amount of piperine or its derivatives present in the formulation of present invention is from about 10% to about 80% of total weight of formulation.

One aspect of the invention relates to the amount of piperine that is to be combined with resveratrol. In some embodiments, the compositions of the invention comprise at least, by weight, about 10%, 20%, 30%, 40%, 50%, 60%, 70%, 80% resveratrol, as well as any percentage that intervenes these specifically listed percentages. In one non-limiting embodiment, the composition comprises about 80% resveratrol and about 16% of the piperine in combination with Tizanidine or its pharmaceutically acceptable salts. The components of the composition can be present in ratios with respect to one another. In one non-limiting embodiment, the compositions can comprise resveratrol and piperine in a ratio of from about 15:1 to about 1:15.

Some aspects of the invention concern formulating the composition for administration to a subject. Accordingly, the compositions of the invention may be formulated to be administered orally, sublingually, intranasally, topically, intravenously, intramuscularly, subcutaneously, buccally, parenterally, intravaginally, rectally, and by inhalation. The compositions may be in a dosage form that includes but is not limited to powders, pills, tablets, pellets, capsules, thin films, solutions, sprays, syrups, linctuses, lozenges, pastilles, chewing gums, pastes, vaporizers, suspensions, solutions, emulsions, ointments, creams, lotions, liniments, gels, drops, topical patches, buccal patches, beads, gummies, gels, sols, injections and the like. The composition may comprise at least one pharmaceutically acceptable excipient. Suitable excipients for use with the invention include, but are not limited to, those disclosed in Remington's Pharmaceutical Sciences, 18th ed. the disclosure of which is incorporated herein by reference in its entirety. The pharmaceutically acceptable excipient can an artificial pharmaceutical carrier. In some aspects of the invention, resveratrol is the only pharmaceutically active component in the composition. In an embodiment, the compositions of the invention may be in the form of a solution, emulsion, or suspension, liposomes and/or micelles.

In an embodiment, the present invention a method of treating a patient suffering from, or susceptible to, a disease that is beneficially treated by tizanidine comprising the step of administering to said patient an effective amount a composition of this invention. Such diseases include, but are not limited to, muscle spasticity; muscle hypertonia; sleep improvement in victims of traumatic brain injury; motor function disorders; suppression of insulin production and the treatment of metabolic disorders resulting from excessive insulin secretion, including type 2 diabetes mellitus, polycystic ovary syndrome, hyperinsulinemia, dyslipidemia, congestive heart disease, glucose intolerance and obesity; pain, notably musculoskeletal pain such as that of the lower back as well as other forms of nociceptive or inflammatory pain; and reduction of somnolence.

In one particular embodiment, the method of this disclosure is used to treat a patient suffering from or susceptible to a disease or condition selected from muscle hypertonia and muscle spasticity associated with multiple sclerosis (MS), spinal cord injury, stroke, cerebral palsy and brain injury; sleep improvement in traumatic brain injury (TBI) victims; and motor function disorders.

In another particular embodiment, the method of this disclosure is used to treat a patient suffering from or susceptible to a disease or condition selected from muscle hypertonia and muscle spasticity associated with multiple sclerosis (MS), spinal cord injury, stroke, cerebral palsy and brain injury. Methods delineated herein also include those wherein the patient is identified as in need of a particular stated treatment. Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to the patient one or more second therapeutic agents. The choice of second therapeutic agent may be made from any second therapeutic agent known to be useful for co-administration with tizanidine. The choice of second therapeutic agent is also dependent upon the disease or condition to be treated.

### EXAMPLES

The following examples are set forth below to illustrate the methods and results according to the disclosed subject matter. These examples are not intended to be 30 inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° c. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1

### Bioavailability of Bio Enhanced Tizanidine Formulation:

The purpose of this study was to assess the effect of co-administration of resveratrol and piperine on the pharmacokinetics of Tizanidine via concentrations in rat plasma after oral and intravenous administration.

### Study Design:

| | | | | | | No. | |
|---|---|---|---|---|---|---|---|
| | | | | | | animals | |
| | | | Dose | | Formulation | (n) | |
| | Treatment | Dose | Volume | | strength | Day | Day |
| Group | (Dose) | (mg/kg) | (mL/kg) | ROA | (mg/mL) | 1 | 5 |
| G1 | Tizanidine | 3 | 3.33 + 3.33 | PO | 0.90 | 6 | 5 |
| | HCl | | (vehicle of | | (Tizanidine HCl) | | |
| | | | Resveratrol) + | | | | |
| | | | 3.33 (vehicle | | | | |
| | | | of piperine) | | | | |
| G2 | Tizanidine | 3 + 44 | 3.33 + 3.33 + 3.33 | PO | 0.90 | 6 | 3 |
| | HCl + | | (vehicle of piperine) | | (Tizanidine HCl) | | |
| | Resveratrol | | | | 13.21 | | |
| | | | | | (Resveratrol) | | |
| G3 | Tizanidine | 3 + 44 + | 3.33 + 3.33 + 3.33 | PO | 0.90 | 6 | 5 |
| | HCl + | 2 | | | (Tizanidine HCl) | | |
| | Resveratrol + | | | | 13.21 | | |
| | piperine | | | | (Resveratrol) | | |
| | | | | | 0.60 | | |
| | | | | | (piperine) | | |
| G4 | Tizanidine | 3 | 3.33 + 3.33 | PO | 0.90 | 6 | 4 |
| | D4 | | (vehicle of | | (Tizanidine | | |
| | | | Resveratrol) + 3.33 | | D4) | | |
| | | | (vehicle of | | | | |
| | | | piperine) | | | | |
| G5 | Tizanidine | 3 + 44 | 3.33 + 3.33 + 3.33 | PO | 0.90 | 6 | 4 |
| | D4 + | | (vehicle of piperine) | | (Tizanidine | | |
| | Resveratrol | | | | D4) | | |
| | | | | | 13.21 | | |
| | | | | | (Resveratrol) | | |
| G6 | Tizanidine | 3 + 44 + | 3.33 + 3.33 + | PO | 0.90 | 6 | 4 |
| | D4 + | 2 | 3.33 | | (Tizanidine | | |
| | Resveratrol + | | | | D4) | | |
| | piperine | | | | 13.21 | | |
| | | | | | (Resveratrol) | | |
| | | | | | 0.60 | | |
| | | | | | (piperine) | | |

### Bioanalysis:

Bioanalysis was performed using a fit-for-purpose LC-MS/MS method for the quantification of tenofovir in rat plasma samples. The calibration curve (CC) for the method consisted of nine non-zero calibration standards along with a double blank and zero standard samples. Study samples were analyzed along with three sets of quality control samples (18 QC samples; low, medium and high QC samples).

### Pharmacokinetic Analysis:

Plasma pharmacokinetic parameters were calculated using the non-compartmental analysis tool of Phoenix software (Version 6.3) and were determined from individual animals in each group. The peak plasma concentration (Cₘₐₓ), time to achieve peak plasma concentration (Tₘₐₓ), the area under the plasma concentration-time curve (AUC₀₋ₜ and AUC_{inf}), AUC Extra (%), elimination half-life (T_{1/2}), clearance (CL), volume of distribution Vd (L/kg) and Mean residence time (MRT) were calculated from intravenous group. The peak plasma concentration (Cₘₐₓ), time to achieve peak plasma concentration (Tₘₐₓ), AUC₀₋ₜ and AUC_{inf}, AUC Extra (%), Mean residence time (MRT) and absolute oral bioavailability (F) were calculated from the oral group.

### Results:

| | | | Group 3 | | | |
|---|---|---|---|---|---|---|
| | | Group 2 | Tizanidine | | Group 5 | Group 6 |
| | Group 1 | Tizanidine | HCl + | Group 4 | Tizanidine | Tizanidine D4 + |
| Group | Tizanidine | HCl + | Resveratrol + | Tizanidine | D4 + | Resveratrol + |
| Formulation | HCl | Resveratrol | piperine | D4 | Resveratrol | piperine |
| Dose (mg/kg)/ | 3/PO | 3 + 44/PO | 3 + 44 + | 3/PO | 3 + 44/PO | 3 + 44 + 2/ |
| ROA | | | 2/PO | | | PO |
| Pk | Plasma | Plasma | Plasma | Plasma | Plasma | Plasma |
| Parameters/Matrix | | | | | | |
| Cₘₐₓ (ng/mL) | 88.4 ± 31.1 | 83.4 ± 47.4 | 143 ± 83.7 | 87.9 ± 48.7 | 104 ± 64.6 | 183 ± 79.5 |
| | (35.2) | (56.8) | (58.4) | (55.4) | (62.2) | (43.5) |
| Tₘₐₓ (hr) ^{[a]} | 0.25 | 0.25 | 0.25 | 0.50 | 0.5 | 0.38 |
| | (0.25-0.5) | (0.25-0.5) | (0.25-0.5) | (0.25-0.5) | (0.25-1.0) | (0.25-1.0) |
| AUCₗₐₛₜ hr | 106 ± 17.7 | 105 ± 36.2 | 208 ± 172 | 177 ± 90 | 252 ± 163 | 359 ± 170 |
| (ng*hr/mL) | (16.7) | (34.6) | (82.7) | (50.7) | (64.6) | (47.4) |
| AUC_{INF} hr | 111 ± 20 | 108 ± 35.1 | 211 ± 176 | 183 ± 85 | 254 ± 162 | 367 ± 164 |
| (ng*hr/mL) | (18) | (32.7) | (83.1) | (46.4) | (63.9) | (44.6) |
| t_{1/2} (hr) | 1.87 ± 1.15 | 1.29 ± 0.675 | 0.923 ± 0.42 | 2.29 ± 2.35 | 1.14 ± 0.373 | 1.5 ± 1.09 |
| | (61.7) | (52.4) | (45.5) | (103) | (32.6) | (72.9) |
| CL_{F}_Obs(PO) | 462 ± 83.5 | 511 ± 178 | 371 ± 223 | 331 ± 171 | 268 ± 163 | 173 ± 115 |
| (mL/min/kg) | (18.1) | (34.7) | (60.2) | (51.7) | (60.8) | (66.6) |
| V_{z/F}_obs(PO) | 71.9 ± 36.1 | 67.1 ± 42.8 | 24.8 ± 11 | 133 ± 181 | 30.1 ± 27.4 | 30.5 ± 41.9 |
| (L/kg) | (50.2) | (63.8) | (44.6) | (136) | (91.3) | (137) |
| Relative | — | 98.8 ± 34.1 | 196.3 ± 162.4 | — | 142 ± 92.2 | 203.3 ± 96.3 |
| Bioavailability | | (34.5) | (82.7) | | (64.7) | (47.4) |
| (F %) | | | | | | |

### Conclusions:

1. Use of Piperine and Resveratrol in combination with Tizanidine HCl increased the relative bioavailability by 96%.

2. Use of Piperine and Resveratrol in combination with Tizanidine D4 increased the relative bioavailability by 103%.

3. The Cmax increased from 88.4 ng/mL to 143 ng/mL, while the AUC improved from 106 ng*hr/mL to 208 ng*hr/mL in the combination group Tizanidine HCl+Resveratrol+piperine

4. The Cmax increased from 87.9 ng/mL to 183 ng/mL, while the AUC improved from 177 ng*hr/mL to 359 ng*hr/mL in the combination group Tizanidine D4+Resveratrol+piperine

### Example 2

### To Compare the Pharmacokinetic Parameters of Tizanidine HCl Vs Tizanidine D4

### Study Design:

| | | | | Dose | | Formulation | |
|---|---|---|---|---|---|---|---|
| | Treatment | Dose | Animal | Volume | | strength | No. |
| Group | (Dose) | (mg/kg) | ID No. | (mL/kg) | ROA | (mg/mL) | animals (n) |
| G7 | Tizanidine | 3 | 01-06 | 3.33 + 3.33 | PO | 0.90 | 6 |
| | HCl | | | (vehicle of | | (Tizanidine | |
| | | | | Resveratrol) + 3.33 | | HCl) | |
| | | | | (vehicle of piperine) | | | |
| G8 | Tizanidine | 3 | 19-24 | 3.33 + 3.33 | PO | 0.90 | 6 |
| | D4 | | | (vehicle of | | (Tizanidine | |
| | | | | Resveratrol) + 3.33 | | D4) | |
| | | | | (vehicle of piperine) | | | |

### Bioanalysis:

Bioanalysis was performed using a fit-for-purpose LC-MS/MS method for the quantification of tenofovir in rat plasma samples. The calibration curve (CC) for the method consisted of nine non-zero calibration standards along with a double blank and zero standard samples. Study samples were analyzed along with three sets of quality control samples (18 QC samples; low, medium and high QC samples).

### Pharmacokinetic Analysis:

Plasma pharmacokinetic parameters were calculated using the non-compartmental analysis tool of Phoenix software (Version 6.3) and were determined from individual animals in each group. The peak plasma concentration (C), time to achieve peak plasma concentration (Tₘₐₓ), the area under the plasma concentration-time curve (AUC₀₋ₜ and AUC_{inf}), AUC Extra (%), elimination half-life (T_{1/2}), clearance (CL), volume of distribution Vd (L/kg) and Mean residence time (MRT) were calculated from intravenous group. The peak plasma concentration (C), time to achieve peak plasma concentration (T), AUC₀₋ₜ and AUC_{inf}, AUC Extra (%), Mean residence time (MRT) and absolute oral bioavailability (F) were calculated from the oral group.

### Results:

| Group | Group 7 | | Group 8 | |
|---|---|---|---|---|
| Formulation | Tizanidine HCl | | Tizanidine D4 | |
| Dose (mg/kg)/ROA | 3/PO | | 3/PO | |
| Pk Parameters/Matrix | Plasma | | Plasma | |
| Cmax (ng/mL) | 88.4 ± 31.1 | (35.2) | 131 ± 131 | (100) |
| Tmax (hr) [a] | 0.25 | (0.25-0.5) | 0.25 | (0.25-0.5) |
| AUClast hr | 106 ± 17.7 | (16.7) | 147 ± 75.2 | (51.2) |
| (ng * hr/mL) | | | | |
| AUCINF hr | 111 ± 20 | (18) | 149 ± 74.3 | (49.7) |
| (ng * hr/mL) | | | | |
| t1/2 (hr) | 1.87 ± 1.15 | (61.7) | 1.11 ± 0.405 | (36.5) |
| CLF_Obs(PO) | 462 ± 83.5 | (18.1) | 405 ± 189 | (46.5) |
| (mL/min/kg) | | | | |
| Vz/F_obs(PO) (L/kg) | 71.9 ± 36.1 | (50.2) | 41.4 ± 33.8 | (81.8) |

| | | |
|---|---|---|
| Relative | — | 138.67% |
| Bioavailability (F %) | | |

### Conclusions:

1. The relative bioavailability of Tizanidine D4 was 138.67% as compared to Tizanidine HCl

2. Deuteriation of Tizanidine increased Cmax from 88.4 ng/mL to 131 ng/mL, while the AUC improved from 106 ng*hr/mL to 147 ng*hr/mL

### Example 3

### Tizanidine Hydrochloride/Resveratrol/Piperine—Tablets

| Sr. No. | Ingredients | Mg/Tablet |
|---|---|---|
| 1. | Tizanidine Hydrochloride | 0.5-50 |
| 2. | Resveratrol | 0.1-500 |
| 3. | Piperine | 10-100 |
| 4. | Glycerol palmitostearate | 10-150 |
| 5. | Microcrystalline cellulose (Avicel PH 101) | 5-100 |
| 6. | Silicon dioxide colloidal (Aerosil 200) | 40-170 |
| 7. | Edetate disodium | 1-7.5 |
| 8. | Sodium starch Glycolate | 30-60 |
| 9. | Magnesium stearate | 3-10 |
| 10. | Talc | 2-5 |

Coating

| | | |
|---|---|---|
| 11. | Opadry ready mix | 10-45 |
| 12. | Purified water | qs |

### Manufacturing Process:

1. Piperine, Resveratrol, microcrystalline cellulose, sodium starch glycolate, edetate disodium were sifted through #30 sieve, Tizanidine Hydrochloride was sifted through #40 sieve and added to a suitable blender.

2. Edetate disodium, Glycerol palmitostearate, Magnesium stearate, colloidal silicon dioxide and talc were sifted through #60 Sieve and added to the blender of step 1.

3. Powders were mixed in a blender for 15 minutes.

4. The blend was then compressed into tablets using suitable tooling using a tablet compression machine.

5. Opadry ready mix was dispersed in purified water to form a coating dispersion.

6. Compressed tablets were coated with the Opadry dispersion using a suitable tablet coating machine.

### Example 4

### Tizanidine Hydrochloride/Resveratrol/Piperine—Capsules

| Sr. No. | Ingredients | Quantity mg/Capsule |
|---|---|---|
| 1. | Tizanidine Hydrochloride | 0.5-50 |
| 2. | Resveratrol | 0.1-500 |
| 3. | Piperine | 10-25 |
| 4. | Pregelatinized corn starch | 25-250 |
| 5. | Colloidal silicon dioxide | 1-15 |
| 6. | Magnesium stearate | 3-15 |
| 7. | Talc | 3-15 |
| 8. | Empty hard gelatin capsule shells | 1 unit |

### Manufacturing Process:

1. Tizanidine Hydrochloride was sifted through #40 sieve, Resveratrol and Piperine were sifted through #30 sieve using a sifter and collected in stainless steel drum.

2. Pregelatinized corn starch, colloidal silicon dioxide and talc were sifted through #60 sieve using a sifter and collected in stainless steel drum.

3. The sieved powders of step 1 &2 were loaded in the blender and mixed for 10 minutes.

4. Magnesium stearate was sifted through #60 sieve using a sifter and the blend of step 3. The blend was further mixed for 5 minutes

5. The blend was then filled in the empty hard gelatin capsule shells using a capsule filling machine.

6. Note: The processing area must be under controlled room temperature and humidity. The limits are RH 50% to 55%, temperature 22° C. to 27° C.

### Example 5

### Tizanidine Hydrochloride/Resveratrol/Piperine—Oral Suspension

| Sr. No. | Ingredients | Quantity mg/5 mL |
|---|---|---|
| 1. | Tizanidine Hydrochloride | 0.5-50.0 |
| 2. | Resveratrol | 0.1-500 |
| 3. | Piperine | 10-50 |
| 4. | Ascorbic acid | 5-15 |
| 5. | Sodium hydroxide | 1.5-5.0 |
| 6. | Edetate disodium (sodium EDTA) | 0.2-2.0 |
| 7. | Saccharin sodium | 0.1-1.0 |
| 8. | Sodium metabisulfite | 1-5 |
| 9. | Alcohol (ethanol, 95%) | 50-100 |
| 10. | Propylene glycol | 75-150 |
| 11. | Sorbitol (70% solution) | 75-150 |
| 12. | Glycerin (glycerol) | 200-350 |
| 13. | Sucrose | 250-400 |
| 14. | Quinoline yellow | 0.01-0.08 |
| 15. | Pineapple flavor | 0.1-0.5 |
| 16. | Purified water | q.s |

### Manufacturing Process:

1. Purified water (˜25% of total quantity required) was added to a manufacturing vessel and heated to 90° C. to 95° C.

2. Required quantity of sucrose was added to the heated water of step 1 under slow mixing (temperature maintained at 90° C. to 95° C.). Mixing was continued for 1 hr.

3. Propylene glycol, sorbitol (70% solution) glycerin was added to the mixture of step 2 and mixed at high speed for 10 minutes.

4. The mixture was allowed to cool to a temperature of 50° C. with continuous mixing at slow speed.

5. Tizanidine Hydrochloride and Resveratrol were sifted through mesh 40 Sieve and added to the solution of step 4 with continuous mixing at high speed for 30 minutes until a uniform suspension was obtained.

6. Ascorbic acid, edetate disodium and sodium metabisulfite were added to the suspension of step 5 with continuous mixing.

7. Piperine was dissolved in ethanol and the solution was added to the suspension of step 5 while continuous stirring at slow speed.

8. Pineapple flavor was dissolved in part quantity of purified water and added to the suspension of step 5 with mixing at slow speed.

9. Sodium hydroxide and Saccharin sodium were dissolved in part quantity of purified water and added to the suspension of step 5 with slow mixing.

10. Quinoline yellow was dissolved in part quantity of purified water and the colour solution was transferred to the suspension of step 5 with slow mixing. Rinsing of the container of colour solution was done with purified water and rinsings added to the solution of step 4. The mixture was mixed at high speed for 5 minutes.

11. Volume makeup was done with purified water and suspension was again mixed for 20 minutes at high speed.

12. pH of the suspension was checked and recorded. If required, pH was adjusted with 10% citric acid or 10% sodium citrate solution.

13. The suspension was then filled in suitable bottles and sealed.

### Example 6

### Tizanidine Hydrochloride/Resveratrol/Piperine—Powder for Oral Suspension (Sachet)

| Sr. No | Ingredients | Qty. mg/unit |
|---|---|---|
| 1. | Tizanidine Hydrochloride | 0.5-50 |
| 2. | Resveratrol | 0.1-500 |
| 3. | Piperine | 5-50 |
| 4. | Polysorbate 80 | 0.25-0.50 |
| 5. | Simethicone | 0.6-1.0 |
| 6. | Xanthan gum | 10-20 |
| 7. | Silicon dioxide | 7.5-12.5 |
| 8. | Titanium dioxide | 15-20 |
| 9. | Sodium benzoate | 6-10 |
| 10. | Cherry flavor, natural and | 2.5-5.0 |
| | artificial | |
| | (microencapsulated) | |
| 11. | Sucrose | q.s.t. 1500 mg |

### Manufacturing Process:

1. Tizanidine Hydrochloride, Resveratrol, Piperine, Xanthan gum, Silicon dioxide, Titanium dioxide, Sodium benzoate, Cherry flavor and sucrose were sifted through #40 sieve.

2. Required quantity of Polysorbate 80 & Semithicone were added on part of sifted sucrose and sifted through #40 sieve.

3. The ingredients of step 1 & 2 were blended in an octagonal blender for 7 minutes.

4. The blend was then filled in sachets & sealed.

### Example 7

### Tizanidine Hydrochloride/Resveratrol/Piperine—Tablets

| Sr. No. | Ingredients | Qty/Tab (mg) |
|---|---|---|
| 1. | Tizanidine Hydrochloride | 0.5-50 |
| 2. | Resveratrol | 0.1-500 |
| 3. | Piperine | 5-75 |
| 4. | Microcrystalline cellulose | 10-35 |
| 5. | Lactose | 50-200 |
| 6. | Crosscarmellose Sodium | 2-10 |
| 7. | Povidone | 3-10 |
| 8. | Polysorbate 80 | 3-10 |
| 9. | Methylene chloride/water | q.s. |
| 10. | Hypromellose | 30-90 |
| 11. | Colloidal Anhydrous silica | 1-5 |
| 12. | Talc | 1-5 |
| 13. | Magnesium Stearate | 1-5 |

Coating

| | | |
|---|---|---|
| 14. | Opadry ready mix | 10-20 |
| 15. | Purified water | Q.S. |

### Manufacturing Process:

1. Tizanidine Hydrochloride, Resveratrol, Piperine, Microcrystalline cellulose, Lactose, Crosscarmellose Sodium were sifted through #i30 sieve and material was loaded in rapid mixer granulator.

2. Dry mixing of the ingredients was carried out for 10 mins.

3. Polysorbate-80 was dissolved in half quantity of methylene chloride/water mixture using overhead stirrer until a clear solution was obtained.

4. Binder solution was prepared by dissolving povidone in remaining quantity of methylene chloride/water under stirring until a clear solution was obtained.

5. Granulation of ingredients of step 2 was carried out using the binder solution of step 4 and Polysorbate 80 solution of step 3 in RMG.

6. The granules were dried and sizing of dried granules was done by passing through #20 sieve.

7. The dried granules were then blended (using octagonal blender) with Hypromellose, silicon dioxide, talc (previously sifted through #60 sieve) followed by lubrication with magnesium stearate.

8. The lubricated granules were then compressed into tablets using a suitable tablet compression machine.

9. The compressed tablets were then coated with the Opadry ready mix dispersion in purified water using a suitable coating machine.

### Example 8

### Tizanidine Hydrochloride/Resveratrol/Piperine—Soft Gelatin Capsules

| Sr. No | Ingredients | Qty. (mg/unit) |
|---|---|---|
| 1. | Tizanidine Hydrochloride | 0.5-50 |
| 2. | Resveratrol | 0.1-500 |
| 3. | Piperine | 25-300 |
| 4. | D-alpha-tocopheryl polyethylene | 50-400 |
| | glycol 1000 succinate (TPGS) | |
| 5. | Polyethylene glycol 400 | 200-350 |

### Manufacturing Process:

1. D-alpha-tocopheryl polyethylene glycol 1000 succinate (TPGS) was added in a suitable stainless steel-jacketed vessel and heated to 50° C. until liquefied.

2. Polyethylene glycol was added (˜80% of the total volume) at 50-C and mix until homogenous solution was obtained.

3. Temperature of the solution was increased to 65° C., and Piperine, Resveratrol, (previously sifted through i60 Sieve) and Tizanidine (Previously sifted through #40 Sieve) were added to the solution and stirred for 60 mins until uniformly dispersed.

4. Add remaining quantity of polyethylene glycol to the dispersion of step 3 and cool to room temperature.

5. Vacuum was applied to remove air entrapped in the dispersion.

6. The suspension was then filled in oblong, brown opaque soft gelatin capsules using a capsule-filling machine.

7. The capsule shells were the dried until the desired moisture levels (3% to 6%) and packed in a suitable container.

### Example 9

### Tizanidine Hydrochloride/Resveratrol/Piperine—Extended Release Tablets

| Sr. No | Ingredients | Qty (mg/tab) |
|---|---|---|
| 1 | Tizanidine Hydrochloride | 10-500 |
| 2 | Resveratrol | 0.1-500 |
| 3 | Piperine | 5-75 |
| 4 | Microcrystalline cellulose | 75-200 |
| 5 | Lactose | 75-200 |
| 6 | Povidone | 4-16 |
| 7 | Corn Starch | 10-45 |
| 8 | Purified water | Q.s. |
| 9 | Hypromellose (HPMC K4M/K15 M/K100 M) | 150-750 |
| 10 | Colloidal silicon dioxide | 1-6 |
| 11 | Talc | 3-12 |
| 12 | Magnesium Stearate | 3-12 |
| 13 | Ready mix opadry | 5-10 |
| 14 | Purified water | Q.s. |

### Manufacturing Process:

1. Binder solution was prepared by dissolving Povidone in purified water.

2. Tizanidine Hydrochloride, Resveratrol, Piperine Microcrystalline cellulose, Lactose and Hypromellose were sifted through #40 mesh sieve.

3. The sifted materials of step 2 were then placed in rapid mixer granulator and dry mixing carried out for 10 mins.

4. The dry mix was further granulated using the binder of step 1.

5. Granules were then dried in a fluid bed drier until the desired LOD was achieved.

6. Sizing of the granules was done by passing the dried granulator through multimill.

7. The dried sized granules were then blended with Colloidal silicon dioxide and talc (pre-sifted through #80 sieve) followed by lubrication with magnesium stearate (pre-sifted through #80 sieve) in an Octagonal blender.

8. The lubricated blend was then compressed into tablets using suitable tooling using a tablet compression machine.

9. The tablets were further film coated using a film coating dispersion. (The film coating dispersion was prepared by dispersing the Opadry dry mix in purified water).

### Example 10

### Tizanidine D4 Tablets (Immediate Release)

| Sr. No. | Ingredients | Qty/Tab (mg) |
|---|---|---|
| 1 | Tizanidine D4 | 0.5-50.0 |
| 2 | Microcrystalline cellulose | 10-200 |
| 3 | Dicalcium Phosphate | 50-200 |
| 4 | Croscarmellose Sodium | 2-20 |
| 5 | Povidone | 3-25 |
| 6 | Polysorbate 80 | 3-10 |
| 7 | Methylene chloride/water | q.s. |
| 8 | Hypromellose | 30-90 |
| 9 | Colloidal silicon dioxide | 1-5 |
| 10 | Talc | 1-5 |
| 11 | Magnesium Stearate | 1-5 |

Coating

| | | |
|---|---|---|
| 12 | Opadry ready mix | 10-20 |
| 13 | Purified water | q.s. |

### Manufacturing Process:

1. Microcrystalline cellulose, Dibasic calcium Phosphate and Croscarmellose sodium were sifted through #35 sieve (500 microns) and collected in a polyelylene bag.

2. Tizanidine D4 was sifted through #60 sieve (250 micron) and collected in a polyelylene bag.

3. The materials of step 1 & 2 were loaded in a suitable mixer granulator (Rapid Mixer Granulator) and mixed for 10 minutes.

4. Polysorbate-80 solution was prepared by dissolving in polysorbate-80 in half quantity of methylene chloride/water under stirring.

5. Binder solution was prepared by dissolving povidone in remaining quantity of methylene chloride/water under stirring. Mix the solution of step to a solution of step 5 under stirring.

6. The dry mix of step 3 was then granulated with the binder solution of step 5 in a rapid mixer granulator till the wet mass of suitable consistency was obtained.

7. The granules were then dried in a fluidized bed drier a until the desired LOD was achieved.

8. Sizing of the dried granules was done by passing through #22 sieve.

9. Blending of the granules of step 8 with Hypromellose (previously sifted through 40 # sieve), silicon dioxide (sifted through #80 sieve) and talc (sifted through #80 sieve) for 10-15 minutes in an octagonal blender.

10. The required quantity of Magnesium stearate was weighed and sifted through #80 sieve and added to the blend of step 9 and blended for 3-7 minutes.

11. The blend of step 10 was then compressed in to tablets using suitable punches using tablet compression machine.

12 The compressed tablets were then film coated with Opadry ready mix using a tablet coating machine.

### Example 11

### Tizanidine D4 Extended Release Tablets

| Sr. No | Ingredients | Qty (mg/tab) |
|---|---|---|
| 1. | 1. | 0.5-50 |
| 2. | 2. | 75-200 |
| 3. | 3. | 75-200 |
| 4. | 4. | 4-16 |
| 5. | 5. | 10-45 |
| 6. | 6. | Q.s. |
| 7. | 7. | 150-750 |
| 8. | 8. | 1-6 |
| 9. | 9. | 3-12 |
| 10. | Magnesium Stearate | 3-12 |
| 11 | Ready mix opadry | 5-10 |
| 12 | Purified water | Q.s. |

Manufacturing Process:

1. Binder solution was prepared by dissolving Povidone in purified water.

2. Tizanidine D4, Microcrystalline cellulose, Lactose and Hypromellose were sifted through #30 mesh sieve.

3. The sifted materials of step 2 were then placed in rapid mixer granulator and dry mixing carried out for 10 mins.

4. The dry mix was further granulated using the binder of step 1.

5. Granules were then dried in a fluid bed drier until the desired LOD was achieved.

6. Sizing of the granules was done by passing the dried granulator through multi-mill.

7. The dried sized granules were then blended with Colloidal silicon dioxide and talc (pre-sifted through #80 sieve) followed by lubrication with magnesium stearate (pre-sifted through #80 sieve) in an Octagonal blender.

8. The lubricated blend was then compressed into tablets using suitable tooling using a tablet compression machine.

9. The tablets were further film coated using a film coating dispersion. (The film coating dispersion was prepared by dispersing the Opadry dry mix in purified water).

### Example 12

### Tizanidine D4—Powder for Oral Suspension

| Sr. No. | Ingredients | Quantity mg/5 ml |
|---|---|---|
| 1. | Tizanidine D4 | 0.5-50 |
| 2. | Xylitol | 100-400 |
| 3. | Citric acid monohydrate | 1-10 |
| 4. | Sucralose | 0.1-7.5 |
| 5. | Xanthan gum | 0.1-1.0 |
| 6. | Talc | 0.25-2.5 |
| 7. | Magnesium stearate | 0.5-2.5 |
| 8. | Colloidal silicon dioxide | 5-10 |
| 9. | Artificial Cherry Flavour | [001]-10 |

### Manufacturing Process:

1. Tizanidine D4 was sifted through sieve #60 and collected in a polyethylene bag.

2. Xylitol and citric acid monohydrate was weighed and sifted through sieve #40 and collected in a suitable polybag.

3. Sucralose, Xanthan gum, Artificial cherry flavor were sifted through sieve #60 and collected in a suitable polyethylene bag.

4. Talc, Magnesium stearate, and colloidal silicon-di-oxide were sifted through sieve #80 and collected in a polyethylene bag.

5. Sifted Tizanidine D4, Xylitol, Citric acid monohydrate, Sucralose, Xanthan gum, Talc and colloidal silicon dioxide were placed in a suitable blender and blended for 10 minutes.

6. Artificial cherry flavor and magnesium stearate was added to the blend of step 5 and further blended for 5 minutes.

7. The blend was then filled in high density HDPE bottles and sealed with CRC caps using induction sealer.

### Example 13

### Tizanidine D4—Soft Gelatin Capsules

| Sr. No. | Ingredient | Qty. mg/unit |
|---|---|---|
| 1. | Tizanidine D4 | 0.5-50 |
| 2. | D-alpha-tocopheryl polyethylene | 150-350 |
| | glycol 1000 succinate (TPGS) | |
| 3. | Polyethylene glycol 400 | 150-300 |
| 4. | Polyethylene glycol 400 | 25-40 |

### Manufacturing Process:

1. D-alpha-tocopheryl polyethylene glycol 1000 succinate (TPGS) was loaded in a suitable stainless steel-jacketed vessel and heated to 50° C. until liquefied.

2. Polyethylene glycol 400 (90%) was heated in a separate stainless steel vessel to 50° C. and added to the liquefied material of step 1 slowly.

3. The ingredients were mixed using a suitable stirrer until homogenous solution obtained.

4. The temperature of the homogenous solution was increased to 65° C., and Tizanidine D4 was added to the solution under constant stirring and dissolved.

5. Remaining quantity of Polyethylene glycol 400 (10%) was added to the solution of step 4 and the solution cooled to room temperature.

6. Vacuum was applied to remove the entrapped air.

7. The mixture was then filled using white opaque soft gelatin capsules (size 12, oblong) using a capsule-filling machine. The capsules were further dried to a moisture level of 3-6% and shell hardness of 7-10 N and packed in a suitable container.

### Example 14

### Tizanidine D4—Suspension (Oral)

| Sr. No. | Ingredients | Quantity mg/mL |
|---|---|---|
| 17. | Tizanidine D4 | 0.5-50.0 |
| 18. | Ascorbic acid | 5-15 |
| 19. | Edetate disodium (sodium EDTA) | 0.2-2.0 |
| 20. | Saccharin sodium | 0.1-1.0 |
| 21. | Sodium metabisulfite (sodium disulfite) | 1-5 |
| 22. | Propylene glycol | 75-150 |
| 23. | Sorbitol (70% solution) | 75-150 |
| 24. | Glycerin (glycerol) | 200-350 |
| 25. | Sucrose | 250-400 |
| 26. | Quinoline yellow | 0.01-0.1 |
| 27. | Pineapple flavor | 0.1-0.5 |
| 28. | Purified water | q.s |

### Manufacturing Process:

1. Purified water (˜25% of total quantity required) was added to a manufacturing vessel and heated to 90° C. to 95° C.

2. Required quantity of sucrose was added to the heated water of step 1 under slow mixing (temperature maintained at 90° C. to 95° C.). Mixing was continued for lhr.

3. Propylene glycol, sorbitol (70% solution) glycerin was added to the mixture of step 2 and mixed at high speed for 10 minutes.

4. The mixture was cooled to a temperature of 50° C. with continuous mixing at slow speed.

5. Tizanidine D4 was added to the solution of step 4 with continuous mixing at high speed for 30 minutes until a uniform suspension was obtained.

6. Ascorbic acid, edetate disodium and sodium metabisulfite were added to the suspension of step 4 with continuous mixing.

7. Pineapple flavor was dissolved in part quantity of purified water and added to suspension of step 4 with continuous mixing.

8. Saccharin sodium were dissolved in part quantity of purified water and added to the suspension of step 4 with continuous mixing.

9. Quinoline yellow was dissolved in part quantity of purified water and the colour solution was transferred to the suspension of step 4 with continuous mixing.

10. Rinsing of the container of colour solution was done with purified water and rinsings added to the Suspension of step 4. The mixture was mixed at high speed for 5 minutes.

11. Volume makeup was done with purified water and solution was again mixed for 20 minutes at high speed.

12. pH of the suspension was checked and recorded (limit: 6.0-8.2). If required, pH was adjusted with 10% citric acid or 10% sodium citrate solution.

13. The suspension was then filled in suitable bottles.

### Example 15

### Tizanidine D4—Extended Release Capsules

| Sr. No | Ingredients | Qty (mg/Capsule) |
|---|---|---|
| | Core pellets | |
| 1 | Tizanidine D4 | 0.5-50 |
| 2 | Microcrystalline cellulose | 75-150 |
| 3 | Lactose | 75-150 |
| 5 | Povidone | 2.5-10 |
| 6 | Purified water | Q.s. |
| | Seal coating | |
| 7 | Hypromellose | 15-40 |
| 8 | Triethyl citrate | 0.25-2.5 |
| 9 | Isopropyl alcohol | QS |
| | Functional coating | |
| 10 | Ethyl cellulose | 20-60 |
| 11 | Triethyl citrate | 0.25-2.5 |
| 12 | Talc | 3-12 |
| 13 | Isopropyl alcohol | Q.s. |
| 14 | Purified water | Q.s. |
| 15 | Empty Hard Gelatin Capsule shell | 1 (Unit) |

### Manufacturing Process:

1. Tizanidine D34, Microcrystalline cellulose, Lactose were sifted through #40 mesh sieve.

2. The sifted materials of step 2 were then placed in planetary mixer and dry mixing was done for 10 minutes.

3. Binder solution was prepared by dissolving Povidone in purified water and wetting of the mass was done by adding the binder solution to the dry mixture blend of step 3.

4. The wet mass of step 3 was then passed through extruder to form cylindrical shaped extrudates.

5. The extrudates were then places in a spheronizer to form pellets.

6. Pellets were then dried in a fluid bed drier until the desired LOD was achieved.

7. Seal coating solution was prepared by dissolving HPMC, PVP and Triethyl citrate in a mixture of Ispropyl alcohol and purified water

8. Pellets were then seal-coated with a seal coating solution using fluid bed processor fitted with a bottom spray assembly.

9. The seal coated pellets were further coated with ethyl cellulose dispersion.

10. Functional coating dispersion was prepared by dissolving ethyl cellulose and triethyl citrate in isopropyl alcohol, talc was dispersed in purified water and this dispersion was then added to the ethlycellulose solution to form a coating dispersion.

11. Seal coated pellets were then coated with ethyl cellulose dispersion using a fluid bed processor fitted with a bottom spray assembly.

12. The coated pellets were then filled in capsules.

### Example 16

### Tizanidine D4 Injection (Lyophilized)

| Sr. No. | Ingredients | Quantity mg/vial |
|---|---|---|
| 1. | Tizanidine D4 | 2.0-50.0 |
| 2. | Sodium chloride, USP | 5-9 |
| 3. | Hydrochloric acid (1 N) for pH adjustment | q.s |
| 4. | Mannitol, USP | 50-500 |
| 5. | Purified water | q.s |

### Manufacturing Process:

1. Dissolve sodium chloride in ˜90% of purified water deaerated by bubbling in of Nitrogen using overhead stirrer.

2. The solution of step 2 was heated to 40° C. to 45° C. and Tizanidine D4 was dissolved under bubbling nitrogen gas and vigorous stirring and subsequent processing was performed by keeping the solution protected from light.

3. Solution of step 2 was allowed to cool to 28′C to 30° C. and mannitol was dissolved in solution of step 2 using overhead stirrer.

4. The pH of the solution was adjusted with 1N Hydrochloric acid.

5. Volume make up was done using purified water solution stirred for 10 minutes.

6. The solution of step 5 was filtered aseptically through 0.22 micron membrane filter.

7. The filtered solution of step 6 was filled aseptically into colorless, sterile glass vials, type I, 20 mL capacity.

8. Vials were subjected to lyophilization process. The lyophilized vials were stoppered with sterile stoppers and sealed with sterile aluminum caps.

### Example 17

### Tizanidine D4 Injection

| Sr. No. | Ingredients | Quantity mg/ml |
|---|---|---|
| 1. | Tizanidine D4 | 2.0-50 |
| 2. | Sodium chloride, USP | 5-9 |
| 3. | Hydrochloric acid (1 N) for pH adjustment | q.s |
| 4. | Purified water | q.s.t 1 ml |

### Manufacturing Process

1. Dissolve sodium chloride in ˜90% of purified water deaerated by bubbling in of Nitrogen using overhead stirrer.

2. The solution of step 2 was heated to 40′C to 45° C. and Tizanidine D4 was dissolved under bubbling nitrogen gas and vigorous stirring and subsequent processing was performed by keeping the solution protected from light.

3. Solution of step 2 was allowed to cool to 28° C. to 30° C. and mannitol was dissolved in solution of step 2 using overhead stirrer.

4. The pH of the solution was adjusted with 1N Hydrochloric acid.

5. Volume make up was done using purified water solution stirred for 10 minutes.

6. The solution of step 5 was filtered aseptically through 0.22 micron membrane filter.

7. The filtered solution of step 6 was filled aseptically into colorless, sterile glass vials, type I and stoppered with sterile stoppers followed by sealing with sterile aluminum caps.

### Example 18

### Tizanidine D4 Nasal Spray

| Sr. No. | Ingredients | Quantity mg |
|---|---|---|
| 1. | Tizanidine D4 | 0.5-50.0 |
| 2. | Polysorbate 80 | 2.5-7.5 |
| 3. | Sodium chloride | 0.1-10 |
| 4. | Citric acid | 0.1-5 |
| 5. | Di sodium Edetate | 0.1-2.5 |
| 6. | Benzalkonium chloride | 0.001-2.5 |
| 7. | Vehicle | Q.s.t. 1 mL |

### Manufacturing Process:

1. Dissolve sodium chloride, Disodium edetate, citric acid, polysorbate 80 in ˜90% of purified water deaerated by bubbling in of Nitrogen using overhead stirrer.

2. The solution of step 1 was heated to 40° C. to 45° C. and Tizanidine D4 was dissolved under bubbling nitrogen gas and vigorous stirring and subsequent processing was performed by keeping the solution protected from light.

3. Solution of step 2 was allowed to cool to 28° C. to 30° C. and mannitol was dissolved in solution of step 2 using overhead stirrer.

4. Required quantity of Benzalkonium chloride was added to the solution of step 3.

5. Volume make up was done using purified water solution stirred for 10 minutes.

6. The solution of step 5 was filtered aseptically through 0.22 micron membrane filter.

7. The filtered solution of step 6 was filled aseptically into sterile containers, sealed and labelled.

### Example 19

### Tizanidine D4 Sublingual Spray

| Sr. No. | Ingredients | Quantity mg |
|---|---|---|
| 1. | Tizanidine D4 | 0.5-50.0 |
| 2. | Propylene glycol | 2.5-25 |
| 3. | Sodium Sulfobutyl ether β cyclodextrin | 10-200 |
| 4. | Methyl Paraben | 0.005-0.05 |
| 5. | Propyl Paraben | 0.005-0.05 |
| 6. | Ethanol | 10-40 |
| 7. | Sucralose | 0.1-10 |
| 8. | Artificial Cherry Flavour | 0.1-4.0 |
| 9. | Purified water | 20-80 |

### Manufacturing Process:

1. Added and dissolved Sodium sulfobutyl ether p cyclodextrin, in purified water using a overhead stirrer.

2. Added and dissolved Tizanidine D4 in the solution of step 1 under continuous stirring.

3. Added and dissolved methyl paraben, Propyl paraben, sucralose and artificial cherry flavor in a mixture of propylene glycol and ethanol using overhead stirrer.

4. Added the solution of step to solution of step 1 under continuous stirring with overhead stirrer.

5. Volume make up was done using purified water solution stirred for 10 minutes.

6. The solution of step 5 was filtered through a suitable filter and filled in a suitable container and container sealed.

### Example 20

### Tizanidine D4—Tizanidine D4 Extended Release Transdermal Patch

| Sr. No. | Ingredients | Quantity mg |
|---|---|---|
| 1. | Tizanidine D4 | 0.5-50.0 |
| 2. | BIO PSA HighTack 7-4301 | 25-75 |
| 3. | BIO PSA MediumTack 7-4201 | 25-75 |
| 4. | Polyvinyl Pyrrolidone | 5-15 |
| 5. | Ascorbyl palmitate | 0.2-2.0 |
| 6. | DL Alpha Tocopherol | 0.1-1.0 |
| 7. | Sodium metabisulphite | 0.0001-1.0 |
| 8. | Ethanol | Q.s. |
| 9. | Heptane | Q.s. |

### Manufacturing Process:

1. Dissolved Tizanidine D4 in required quantity of Ethanol.

2. Dissolve Polyvinyl pyrrolidone, sodium metabisulphite (as 10% solution), ascorbyl palmitate, DL Alpha tocopherol in required quantity of ethanol.

3. Dissolve BIO PSA HighTack 7-4301 and BIO PSA MediumTack 7-4201 in required quantity of heptane.

4. The solutions of step 1 & 3 were added to the solution of step 2 and stirred until a homogenous dispersion was obtained.

5. The dispersion was applied to a suitable polyester release lining using a suitable doctor blade and the solvents were continuously removed in a drying oven at a temperature of about 80° C. for approximately 30 minutes and a drug containing adhesive matrix having desired coating weight was obtained.

6. The dried film was laminated polyester-type backing film.

7. The individual plasters of the desired sizes were punched out of the completed laminates and sealed in the bags under nitrogen.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to a “cosolvent” refers to a single cosolvent or to combinations of two or more cosolvents, and the like.

We claim:

1. A method for enhancing the bioavailability of tizanidine comprising co-administration of therapeutically effective amount of tizanidine or its pharmaceutically acceptable salts, esters, solvates, polymorphs, stereoisomers or mixtures thereof with an effective amount of resveratrol or its derivatives thereof and an effective amount of bioenhancer in the form of a formulation.

2. The method as claimed in claim 1, wherein the tizanidine can be can be deuterated tizanidine.

3. The method as claimed in claim 1, wherein the tizanidine or its pharmaceutically acceptable salts, esters, solvates, polymorphs, stereoisomers or mixtures thereof, deuterated tizanidine is present in an amount from about 2% to about 40% by weight of total formulation.

4. The method as claimed in claim 1, wherein the amount of resveratrol is from about 10% to about 80% of total weight of formulation.

5. The method as claimed in claim 1, wherein the bioenhancer is selected from piperine, garlic, Carum carvi, Currinum cyrrinurn lysergol, naringin, quercetin, niaziridin, glycyrrhizin, stevia, cow urine, distillate ginger, or any combination thereof.

6. The method as claimed in claim 1, wherein the bioenhancer is selected from synthetically prepared piperine, extract from black pepper and extract from Piper longum.

7. The pharmaceutical formulation of claim 5, wherein bioenhancer is selected from tetrahydropiperine, cis-piperine, transpiperine, cis-trans piperine, trans, cis-piperine, cis, cis-piperine, trans, transpiperine or a combination thereof.

8. The pharmaceutical formulation of claim 1, wherein the piperine or its derivatives is present at an amount from about from about 10% to about 80% of total weight of formulation.

9. The method as claimed in claim 1 wherein the resveratrol and piperine are present in a ratio of from about 15:1 to about 1:15.

10. The method as claimed in claim 1, wherein tizanidine or its pharmaceutically acceptable salts, esters, solvates, polymorphs, stereoisomers or mixtures thereof, or deuterated form of tizanidine can be co-administered simultaneously, separately or sequentially with resveratrol and bioenhancer to the patient in need thereof.

11. The method as claimed in claim 1, wherein the tizanidine or its pharmaceutically acceptable salts, esters, solvates, polymorphs, stereoisomers or mixtures thereof, or deuterated can be co-administered with resveratrol and bioenhancer in the form of a dosage form, selected from, tablet, mini-tablet, granules, sprinkles, capsules, sachets, powders, pellets, disintegrating tablets, dispersible tablets, solution, suspension, emulsion, lyophilized powder.

12. The method as claimed in claim 1, wherein the tizanidine or its pharmaceutically acceptable salts, esters, solvates, polymorphs, stereoisomers or mixtures thereof, or deuterated is co-administered simultaneously, separately or sequentially with resveratrol and bioenhancer by a kit.

13. The method as claimed in claim 1, wherein the bioavailability of tizanidine is increased from about from about 10% to about 100%.

14. The method of claim 1, wherein the dose of tizanidine is decreased from about method of about 5% to about 95% as compared to standard dose of tizanidine.

15. The method as claimed in claim 1, wherein the tizanidine or its pharmaceutically acceptable salts, esters, solvates, polymorphs, stereoisomers or mixtures thereof, or deuterated tizanidine is co-administered with resveratrol and bioenhancer for the treatment of muscle spasms.

16. The formulation for enhancing the bioavailability of tizanidine comprising:
a. therapeutically effective amount of tizanidine or or its pharmaceutically acceptable salts, esters, solvates, polymorphs, stereoisomers or mixtures thereof, or deuterated tizanidine,
b. therapeutically effective amount of resveratrol or its derivatives,
c. therapeutically effective amount of piperine or its derivatives,
d. optionally pharmaceutically acceptable excipients.
